(19) United States
(12) Patent Application Publication
(10) Pub. No.: US 2012/0283325 A1
(43) Pub. Date: Nov. 8, 2012

(54) EXCIPIENT COMPATIBILITY WITH EZATIOSTAT

(75) Inventor: Robert T. Lum, Palo Alto, CA (US)

(73) Assignee: Telik, Inc.

(21) Appl. No.: 13/449,432

(22) Filed: Apr. 18, 2012

Related U.S. Application Data
(60) Provisional application No. 61/482,143, filed on May 3, 2011.

(51) Int. Cl. A61K 31/216; A61P 7/00

(52) U.S. Cl. 514/533

(57) ABSTRACT

Disclosed herein is the surprising and unexpected discovery that mannitol inhibits the growth of impurities and enhances shelf life of ezatiostat hydrochloride formulations.

### CROSS-REFERENCE TO RELATED APPLICATIONS

This application claims the benefit under 35 U.S.C. §119(e) of U.S. Provisional Application No. 61/482,143, filed May 3, 2011, the entire disclosure of which is incorporated herein by reference.

### FIELD OF THE INVENTION

This invention relates to compatibility study of ezatiostat hydrochloride with a wide variety of pharmaceutically acceptable excipients for formulation purposes.

### STATE OF THE ART

Ezatiostat and its salts are disclosed in U.S. Pat. No. 5,763,570. Ezatiostat has the IUPAC chemical name of ethyl (2S)-2-amino-5-[[(2R)-3-benzylsulfanyl-1-[[(JR)-2-ethoxy-2-oxo-1-phenylethyl]amino]-1-oxopropan-2-yl]amino]-5-oxopentanoate.

It has been discovered that ezatiostat salts and, in particular, the hydrochloride salt, can be formed as a crystalline ansolvate, referred to as form D, which is disclosed in U.S. Patent Application Publication US-2011-0301088-A1, the contents of which are incorporated herein by reference in its entirety.

Ezatiostat hydrochloride (USAN) has the molecular weight of 566.1, the trademark of Telintra®, and the CAS registry number of 286942-97-0. Ezatiostat hydrochloride has been evaluated for the treatment of myelodysplastic syndrome (MDS), in a Phase I-IIa study using a liposomal formulation (U.S. Pat. No. 7,029,695), as reported at the 2005 Annual Meeting of the American Society for Hematology (Abstract #2250) and by Raza et al. in Journal of Hematology & Oncology, 2:20 (published online on 13 May 2009); and in a Phase I study using a tablet formulation, as reported at the 2007 Annual Meeting of the American Society for Hematology (Abstract #1454) and by Raza et al. in Blood, 113:6533-6540 (prepublished online on 27 Apr. 2009), and in a single patient case report by Quddus et al. in Journal of Hematology & Oncology, 3:16 (published online on 23 Apr. 2010). The entire disclosures of each of the patents and publications referred to in this application are incorporated into this application by reference.

For the clinical formulation of ezatiostat hydrochloride for treatment of MDS, a variety of pharmaceutically acceptable excipients were evaluated. It was surprisingly and unexpectedly found that one such pharmaceutically acceptable excipient, namely, mannitol, inhibited the growth of impurities in the ezatiostat hydrochloride formulation.

### SUMMARY OF THE INVENTION

This invention is directed to the surprising and unexpected discovery that mannitol, a pharmaceutically acceptable excipient, inhibited the formation of degradation products in the ezatiostat hydrochloride formulation.

Accordingly, in one embodiment, this invention is directed to a method for enhancing shelf life of ezatiostat hydrochloride by formulating ezatiostat hydrochloride with an effective amount of mannitol as at least one of the pharmaceutically acceptable excipients.

In another embodiment, this invention is directed to a method for inhibiting formation of degradation products in ezatiostat hydrochloride formulation by formulating ezatiostat hydrochloride with an effective amount of mannitol as at least one of the pharmaceutically acceptable excipients.

As provided in the examples below, it has been surprisingly found that the pharmaceutically acceptable excipient, mannitol, inhibited the formation of degradation products and enhanced the shelf life of ezatiostat hydrochloride formulation.

### DETAILED DESCRIPTION

This invention is directed to the stability study of various pharmaceutically acceptable excipients with ezatiostat hydrochloride. However, prior to describing this invention in more detail, the following terms will first be defined.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “about” when used before a numerical designation, e.g., temperature, time, amount, and concentration, including range, indicates approximations which may vary by (+) or (−) 15%, 10%, 5% or 1%.

The singular forms “a,” “an,” and “the” and the like include plural referents unless the context clearly dictates otherwise. Thus, for example, reference to “a compound” includes both a single compound and a plurality of different compounds.

“Room temperature” refers to (22±5)° C.

“Effective amount” of mannitol refers to an amount of mannitol used for formulation of ezatiostat hydrochloride that inhibits the formation of degradation products. Preferably, the amount is from about 13% to about 50% by weight of the formulation.

The term “shelf life” refers to the length of time that a pharmaceutical formulation can be stored under required conditions while maintaining the level of impurities in the formulation within acceptable levels. Typically, the impurities arise from degradation of the active ingredient in the formulation. Accordingly, in such cases, a reduced shelf life correlates with a shorter period during which the amount of active in the formulation is maintained at the desired level.

The term “enhancing shelf life” refers to those formulations wherein the length of time during which the amount of impurities is maintained within specifications during storage under ambient conditions is extended as compared to similar formulations.

Often times, shelf life is determined under accelerated conditions such as higher temperature and/or humidity. The results are then correlated to ambient conditions using well known correlation tables.

### Methods

This invention provides methods for enhancing shelf life of ezatiostat hydrochloride as well as methods for inhibiting formation of degradation products in ezatiostat hydrochloride formulation.

In one embodiment, this invention provides a method for enhancing shelf life of ezatiostat hydrochloride by formulating ezatiostat hydrochloride with an effective amount of mannitol as at least one of the pharmaceutically acceptable excipients. In a further embodiment, shelf life enhancement is at least 8 weeks at 40° C. In another embodiment, mannitol and ezatiostat hydrochloride are in a ratio of about 1:1 to about 1:6 by weight. In a further embodiment, mannitol and ezatiostat hydrochloride are in a ratio of about 1:1. In a further embodiment, mannitol and ezatiostat hydrochloride are in a ratio of about 1:5.7. In a further embodiment, the method further comprises one or more pharmaceutically acceptable excipients selected from the group consisting of microcrystalline cellulose, croscarmellose sodium, crospovidone, povidone K-29/32, HPMC E5 premium, colloidal silicon dioxide, and magnesium stearate. In a preferred embodiment, the pharmaceutically acceptable excipients are colloidal silicon dioxide, and magnesium stearate. In a further embodiment, the method comprises a ratio of ezatiostat hydrochloride to the pharmaceutically acceptable excipients of 3.3:1.

In another embodiment, this invention provides a method for inhibiting formation of degradation products in ezatiostat hydrochloride formulation by formulating ezatiostat hydrochloride with an effective amount of mannitol as at least one of the pharmaceutically acceptable excipients. In a further embodiment, the formation of degradation products is inhibited for at least 8 weeks. In another embodiment, mannitol and ezatiostat hydrochloride are in a ratio of about 1:1 to about 1:6 by weight. In a further embodiment, mannitol and ezatiostat hydrochloride are in a ratio of about 1:1. In a further embodiment, mannitol and ezatiostat hydrochloride are in a ratio of about 1:5.7. In a further embodiment, the method further comprises one or more pharmaceutically acceptable excipients selected from the group consisting of microcrystalline cellulose, croscarmellose sodium, crospovidone, povidone K-29/32, HPMC E5 premium, colloidal silicon dioxide, and magnesium stearate. In a preferred embodiment, the pharmaceutically acceptable excipients are colloidal silicon dioxide, and magnesium stearate. In a further embodiment, the method comprises a ratio of ezatiostat hydrochloride to the pharmaceutically acceptable excipients of 3.3:1.

### EXAMPLES

The following examples describe the excipient compatibility study for ezatiostat hydrochloride. Unless otherwise stated, all temperatures are in degrees Celcius (° C.) and the following abbreviations have the following definitions:
HPMC=Hydroxypropyl methyl cellulose
RH=Relative humidity
RSD=Relative standard deviation
RRT=Relative retention time

### Example 1

### Formulations of Ezatiostat Hydrochloride

Two different formulations comprising ezatiostat hydrochloride were prepared by mixing ezatiostat hydrochloride with the each of the excipient mixtures 1 and 2 in a 3.3:1 ratio. Table 1 provides the different ingredients used in the excipient mixtures 1 and 2.

TABLE 1
Excipient mixtures for ezatiostat hydrochloride formulations

| | Excipient | Excipient |
|---|---|---|
| Ingredient | mixture 1 (mg) | mixture 2 (mg) |
| Microcrystalline cellulose | 88.25 | |
| (Avicel PH 112) | | |
| Mannitol granular | | 88.25 |
| (Mannogem granular 2080) | | |
| Croscarmellose sodium | 32.5 | |
| Crospovidone | | 32.5 |
| Povidone K-29/32 | | 19.5 |
| HPMC E5 Premium | 19.5 | |
| Colloidal silicon dioxide | 3.25 | 3.25 |
| Magnesium stearate | 6.5 | 6.5 |

Table 2 provides the actual quantities of excipient mixtures used in preparing the two different formulations. Specifically, formulation 1 was prepared by mixing 75 mg of excipient mixture 1 with 250 mg of ezatiostat hydrochloride, whereas formulation 2 was prepared by mixing 75 mg of excipient mixture 2 with 250 mg of ezatiostat hydrochloride as shown in Table 2.

TABLE 2
Two formulations for ezatiostat hydrochloride

| | Absolute Ratio of | | |
|---|---|---|---|
| | ezatiostat | Active | Excipient mixtures |
| Excipient | hydrochloride:Excipients | (mg) | (mg) |
| Formulation 1 | 3:3:1 | 250 | 75 |
| Formulation 2 | 3:3:1 | 250 | 75 |

### Example 2

### Binary Mixtures of Ezatiostat Hydrochloride with Various Excipients

Further, ezatiostat hydrochloride was mixed with each of the excipients mentioned in Table 3. The quantity of excipient used was determined by the desired properties of the resulting formulation. These blends were prepared in scintillation vials which were screw Teflon capped and stored at 40° C. and 75% RH. The results of those binary mixtures are shown below in Table 3.

TABLE 3
Excipients in binary mixtures with ezatiostat hydrochloride

| | Function of | Absolute Ratio of |
|---|---|---|
| Excipient Name | Excipient | Ezatiostat•HCl:Excipient |
| Avicel PH 112 | Diluent | 1:1 |
| Mannitol granular 2080 | Diluent | 1:1 |
| Croscarmellose sodium | Disintegrant | 1:1 |
| Crospovidone | Disintegrant | 1:1 |
| Povidone K-29/32 | Binder | 1:1 |
| HPMC E5 Premium | Binder | 1:1 |
| Colloidal silicon dioxide | Glidant | 5:1 |
| Magnesium stearate | Lubricant | 5:1 |

### Example 3

### Potency Studies of Binary Mixtures and Formulations of Ezatiostat Hydrochloride

The potency results of ezatiostat hydrochloride in the binary mixtures of Table 3, Formulations 1 and 2 of Table 2, and ezatiostat hydrochloride alone stored at 40° C. and 75% RH are shown in Table 4.

TABLE 4
Potency results at 40° C. and 75% RH

| Time points* | T = 0 weeks | T = 4 weeks | T = 8 weeks |
|---|---|---|---|
| Avicel PH 112 | 102.5% | 100.7% | 102.9% |
| Mannitol granular 2080 | 101.6% | 102.2% | 102.7% |
| Croscarmellose sodium | 100.8% | 100.1% | 100.3% |
| Crospovidone | 102.7% | 101.6% | 103.7% |
| Povidone K-29/32 | 103.1% | 102.2% | 96.5% |
| HPMC E5 Premium | 100.9% | 104.0% | 96.5% |
| Colloidal silicon dioxide | 101.5% | 100.6% | 101.1% |
| Magnesium stearate | 101.9% | 101.9% | 101.5% |
| Formulation 1 | 101.1% | 102.3% | 101.5% |
| Formulation 2 | 102.4% | 101.9% | 102.1% |
| Ezatiostat•HCl | 102.1% | 102.0% | 100.4% |

*The first eight entries in Table 4 refer to the binary mixtures of ezatiostat hydrochloride with the excipient in a ratio as indicated in Table 3.

The potency assay data show little change over eight weeks at storage conditions of 40° C. and 75% RH for formulations 1 and 2 and ezatiostat hydrochloride. The potency assay results for all binary mixtures were acceptable over eight weeks at the storage conditions of 40° C. and 75% RH, although Povidone K-29/32 and HPMC E5 Premium showed relatively greater potency change compared to the other excipients. In conclusion, ezatiostat hydrochloride is compatible with all the excipients investigated above.

### Example 4

### Degradation Studies of Binary Mixtures and Formulations of Ezatiostat Hydrochloride

The degradation products were evaluated for each sample using HPLC at initial, 4 weeks, and 8 weeks period. For the initial and 4 week samples, the HPLC autosampler temperature was set at ambient but it was set at 5° C. for samples tested at 8 weeks. The autosampler temperature was lowered due to an observation that individual impurities were increasing during the HPLC run. The degradation data is shown in Table 5.

TABLE 5
Degradation of binary mixtures and formulations of ezatiostat hydrochloride over time

| | Time | RS(% Area) | | | | | | |
|---|---|---|---|---|---|---|---|---|
| | point | RRT | RRT | RRT | RRT | RRT | RRT | |
| Excipient* | (weeks) | 0.53 | 0.74 | 0.81 | 0.82 | 0.88 | 1.24 | Total |
| Avicel PH 112 | 0 | | 0.15% | | | 0.09% | | 0.24% |
| | 4 | | 0.30% | | | 0.35% | | 0.65% |
| | 8 | | 0.06% | | | 0.46% | | 0.52% |
| Mannitol | 0 | | 0.16% | | | 0.09% | | 0.25% |
| granular 2080 | 4 | | 0.31% | | | 0.26% | | 0.57% |
| | 8 | | 0.06% | | | 0.27% | | 0.33% |
| Croscarmellose | 0 | | 0.15% | | | 0.10% | | 0.26% |
| sodium | 4 | | 0.29% | | | 0.49% | | 0.78% |
| | 8 | | 0.07% | | | 0.67% | | 0.73% |
| Crospovidone | 0 | | 0.18% | | | 0.10% | | 0.28% |
| | 4 | | 0.33% | 0.06% | 0.07% | 0.29% | | 0.74% |
| | 8 | | 0.08% | 0.13% | 0.15% | 0.35% | | 0.71% |
| Povidone K- | 0 | | 0.19% | | | 0.11% | | 0.30% |
| 29/32 | 4 | | 0.35% | 0.08% | 0.09% | 0.36% | | 0.88% |
| | 8 | | 0.10% | 0.16% | 0.18% | 0.74% | 0.08% | 1.3% |
| HPMC E5 | 0 | 0.07% | 0.21% | | | 0.12% | | 0.39% |
| Premium | 4 | | 0.33% | | | 0.28% | | 0.61% |
| | 8 | | 0.07% | | | 0.42% | | 0.49% |
| Colloidal | 0 | | 0.21% | | | 0.12% | | 0.33% |
| silicon | 4 | | 0.36% | | | 0.21% | | 0.57% |
| dioxide | 8 | | 0.09% | | | 0.48% | | 0.57% |
| Magnesium | 0 | | 0.22% | | | 0.12% | | 0.34% |
| stearate | 4 | | 0.36% | | | 0.29% | | 0.66% |
| | 8 | | 0.07% | | | 0.42% | | 0.49% |
| Formulation 1 | 0 | | 0.23% | | | 0.12% | | 0.35% |
| | 4 | | 0.38% | | | 0.41% | | 0.79% |
| | 8 | | 0.07% | | | 0.51% | | 0.58% |
| Formulation 2 | 0 | | 0.25% | | | 0.13% | | 0.37% |
| | 4 | | 0.40% | | | 0.27% | | 0.67% |
| | 8 | | 0.07% | | | 0.26% | | 0.38% |
| Ezatiostat•HCl | 0 | | 0.13% | | | 0.09% | | 0.22% |
| | 4 | | 0.28% | | | 0.27% | | 0.55% |
| | 8 | | 0.07% | | | 0.51% | | 0.58% |

*The first eight entries in Table 5 refer to the binary mixtures of ezatiostat hydrochloride with the excipient in a ratio as indicated in Table 3.

The inconsistent results obtained at four weeks were attributed to higher temperature used during HPLC. For this reason, the data at four weeks is not probative for the underlying study.

As can be seen from the data in Table 5, the impurities at 8 weeks at RRT 0.88 in the binary mixture comprising mannitol were 0.27% and the impurities at 8 weeks at RRT 0.88 in Formulation 2 comprising mannitol were 0.26%, whereas the impurities in ezatiostat hydrochloride at 8 weeks at RRT 0.88 were 0.51%. Thus, the % area of the impurity seen at RRT 0.88 with formulations comprising mannitol was half of that seen in ezatiostat hydrochloride alone. On the other hand, in the binary mixture of Povidone K-29/32 the impurities at 8 weeks at RRT 0.88 were 0.74% which was much greater than the impurities in ezatiostat hydrochloride alone. For total impurities, the results were similar to the trend seen with the individual impurity at RRT 0.88, with % area for the total impurities being lower in the binary mixture comprising mannitol and in Formulation 2 than in ezatiostat hydrochloride alone. Thus, mannitol inhibited the formation of degradation products in the ezatiostat hydrochloride formulation.

1. A method for enhancing shelf life of ezatiostat hydrochloride by formulating ezatiostat hydrochloride with an effective amount of mannitol as at least one of the pharmaceutically acceptable excipients.

2. A method for inhibiting formation of degradation products in ezatiostat hydrochloride formulation by formulating ezatiostat hydrochloride with an effective amount of mannitol as at least one of the pharmaceutically acceptable excipients.

3. The method of claim 1, wherein shelf life enhancement is at least 8 weeks.

4. The method of claim 2, wherein the formation of degradation products is inhibited for at least 8 weeks.

5. The method of claims 1 or 2, wherein mannitol and ezatiostat hydrochloride are in a ratio of about 1:1 to about 1:6.

6. The method of claim 5, wherein mannitol and ezatiostat hydrochloride are in a ratio of about 1:1.

7. The method of claim 5, wherein mannitol and ezatiostat hydrochloride are in a ratio of about 1:5.7.

8. The method of claims 1 or 2, wherein ezatiostat hydrochloride is further formulated with one or more pharmaceutically acceptable excipients selected from the group consisting of microcrystalline cellulose, croscarmellose sodium, crospovidone, povidone K-29/32, HPMC E5 premium, colloidal silicon dioxide, and magnesium stearate.

9. The method of claim 8, wherein the pharmaceutically acceptable excipients are crospovidone, povidone K-29/32, colloidal silicon dioxide, and magnesium stearate.

10. The method of claim 9, wherein the method comprises a ratio of ezatiostat hydrochloride to the pharmaceutically acceptable excipients of 3.3:1.
